(19) United States
(12) Patent Application Publication
(10) Pub. No.: US 2012/0121562 A1
(43) Pub. Date: May 17, 2012

(54) NUTRITIONAL COMPOSITION FOR BREAST-FED INFANTS OR PETS WITH PROBIOTICS AND SELECTED NUTRIENTS

(75) Inventors: Gabriela Bergonzelli Degonda, Bussigny (CH); Isabelle Bureau-Franz, Epalinges (CH); Clara Lucia Garcia-Rodenas, Forel (CH); Corinne Magliola, Pully (CH)

(73) Assignee: NESTEC S.A., Vevey (CH)

(21) Appl. No.: 13/387,405

(22) PCT Filed: Jul. 28, 2010
(86) PCT No.: PCT/EP10/60973

§ 371 Date: Jan. 27, 2012

(30) Foreign Application Priority Data
Jul. 31, 2009 (EP) 09166969.7

(51) Int. Cl. A61K 35/74; A61P 1/06; A61P 1/08; A61P 37/08; A61P 17/00; A61P 27/02; A61P 25/28; A61P 1/00; A61P 25/00; A61P 1/12; A61P 3/00; A61P 3/02; A61P 31/00

(52) U.S. Cl. 424/93.44; 424/93.45; 424/93.4; 424/93.48

(57) ABSTRACT

The present invention relates to a nutritional supplement compositions for breast-fed infants or pets which comprise at least one probiotic and at least one nutrient. The compositions are aimed at preserving exclusive breast feeding in infants. The compositions of the invention are also for use in improving the health of breast-fed infants. A method of preparing a nutritional supplement composition for breast-fed infants is also provided.

### FIELD OF THE APPLICATION

The present invention relates to nutritional supplement compositions for breast-fed infants or pets which comprise at least one probiotic and at least one nutrient. The compositions are aimed at preserving exclusive breast feeding in infants. The compositions of the invention are also for use in improving the health of breast-fed infants. A method of preparing a nutritional supplement composition for breast-fed infants is also provided.

### BACKGROUND ART

It has been well established that mother's milk is recommendable for all new-born infants. In the case where the mother cannot breast-feed for medical or other reasons, infant formulas have been developed which try to match as closely the composition of human milk.

However, even in the cases where the mother exclusively breastfeeds an infant, the mother's milk may still be lacking in certain important nutrients, depending on external factors such as the mother's diet, environment, medical condition etc.

It has been established that lactating women are more likely to suffer from specific nutrient deficiencies than from a general shortage of dietary energy or protein. Also, micronutrient deficiencies are more likely to affect breast milk composition and the development and nutritional status of the infant.

A solution proposed in an article entitled “Maternal micronutrient malnutrition: effects on breast milk and infant nutrition and priorities for intervention” by Lindsay H Allen, in SCN News, 11, mid-1994, has been to supplement the lactating mother's diet with the required nutrients in order to adjust the level of micronutrients in the milk composition.

However, this solution is not always convenient and rather indirect. In addition, the breast milk concentration of some nutrients is difficult to restore by supplementation of the mother diet during lactation. Furthermore, other beneficial ingredients to infants such as probiotics cannot reach the infant via supplementation of the mother's diet. In this respect, infant supplements comprising probiotics have been developed and are known for example from WO 2007/142596.

It has now been found that by supplementing the breast milk itself, without having to resort to traditional infant formulas, a number of advantages can be derived for the infant and the mother.

### OBJECT OF THE PRESENT INVENTION

The object of the present invention is therefore to provide a supplement composition which alleviates some of the deficiencies which can occur in exclusively breast-fed infants. In some aspects the invention compensates for suboptimal breast milk content of some nutrients.

### SUMMARY OF THE PRESENT INVENTION

The object is solved by means of the independent claims. The dependent claims further develop the central idea of the invention.

In a first aspect, the present invention relates to a nutritional supplement composition for breast-fed infants or pets comprising at least one probiotic and at least one nutrient selected from the most variable nutrients in human breast milk and pet milk, respectively.

The use of a composition of the invention for preserving exclusive breast feeding in infants also forms part of the invention.

A third aspect of the invention relates to a composition of the invention for use in improving the health of breast-fed infants.

Finally, the invention also pertains to a method of preparing a nutritional supplement composition for breast-fed infants or pets comprising the steps of:
selecting at least one nutrient from the most variable nutrients in human breast milk, and
incorporating said at least one nutrient in a nutritional matrix to form a nutritional supplement composition.

### DETAILED DESCRIPTION OF THE INVENTION

The present invention relates to a nutritional supplement composition.

By “supplement composition” is meant a composition which is to be supplemented to a complete nutritional diet. The supplement composition is thus not intended to provide a complete, balanced nutritional diet.

The composition is intended for breast-fed infants or pets. By “infants” is meant babies from 0 months old up to 4 years, typically 0 months to 18 months. Similarly and by extension the concept of the invention can be applied to pets, such as e.g. cats and dogs.

The composition is preferably aimed for infants which are exclusively breast fed. By “exclusively breast fed” is meant that the infant derives its full energy requirements in terms of carbohydrate, protein and fat exclusively from a mother's milk. Such infants do not benefit from the supplementation in various nutrients brought by the infant formula or other non-breast-milk components of the diet. Hence it appears critical for these “exclusively breast fed infants” to compensate for the variability of the human breast-milk by an adequate supplementation, especially in those nutrients that are the most variable in human breast-milk. In another embodiment, and by extension of the same principle, the invention is targeted at infants receiving 50% or more, or 80% or more, of their daily caloric diet from human breast milk.

Preferably, the supplement composition of the invention provides a caloric dose between more than 0 and 50 Kcal/day, preferably between 1 and 20 Kcal/day, more preferably between 2 and 10 Kcal/day.

The supplement composition of the invention may provide a fat dose between 0 and 6 g/day, preferably between 0.01 and 3 g/day, more preferably between 0.05 and 0.6 g/day.

The supplement composition of the invention may provide a carbohydrate dose between 0 and 4 g/day, preferably between 0.01 and 2 g/day, more preferably between 0.02 and 0.4 g/day.

Typically, the supplement composition provides a protein dose of less than 1.5 g/100 kcal, or less than 1 g/100 kcal, preferably less than 0.5 g/100 kcal.

Preferably, the composition does not contain any protein. In one embodiment the composition however comprises peptides, preferably small peptides of 2 to 25 amino acids length, and/or free amino-acids.

The composition of the invention can have a fat value of more than 5 g fat/100 kcal, preferably more than 8 g fat/100 kcal or even preferably more than 10 or 15 g/100 kcal. In one embodiment the fat content comprises more than 50% or more than 80% of the caloric value of the composition. Such high fat content primarily originates from the oil matrix.

Alternatively, invention can have a fat value of less than 1 g fat/100 kcal, preferably less than 0.5 g fat/100 kcal.

In one embodiment the caloric density of the composition is much above the usual caloric density for infant formula (the invention thus is a supplement, comprising for example a oil-calorie rich-matrix). In one embodiment the caloric density of the composition is much below the usual caloric density for infant formula (the invention thus is a supplement, having low caloric density but highly relevant nutrient content). In one embodiment the caloric density is 1.3 kcal/g or less, 1 kcal/g of composition or less, 0.5 kcal/g of composition or less, 0.25 kcal/g of composition or less. In one embodiment (liquid composition) the caloric density of the composition is 1.5 kcal/ml or more, 3 kcal/ml or more, or 5 kcal/ml or more.

Preferably, the composition is non-dairy. In one embodiment the composition is substantially free of casein and/or of whey protein (less than 5%, less than 1% or less than 0.1% of each).

The composition of the invention thus differentiates itself from infant formulas which tend to provide infants with a complete, balanced nutritional diet. In this respect, the present composition is to be used as a supplement to breast milk.

According to the invention, the composition comprises at least one probiotic and at least one nutrient selected from the most variable nutrients in human breast milk.

By “the most variable nutrients in human breast milk” is meant those nutrients which vary the most in the human milk composition when comparing human milk composition across a population of women. In particular, several scientific articles describe those nutrients that are the most variable in human milk (for example see “Maternal micronutrient malnutrition effects on breast milk and infant nutrition and priorities for intervention, by Lindsay H Allen et al, SCN news N° 11, 1994, United Nations, administrative committee on coordination, Subcommittee on Nutrition).

By choosing nutrients which are the most variable in human breast milk, the composition of the invention is able to better cover for deficiencies occurring in breast milk. Thus, a more efficient supplement can be produced.

Typically, these nutrients are selected from vitamins, provitamins, carotenoids, polyunsaturated fatty acids, long chain polyunsaturated fatty acids, minerals and amino acids. In a particular embodiment, these nutrients can also include peptides.

Thus, the nutrient may be selected from any of docosahexaenoic acid (DHA), alpha-linolenic acid, carotenoids (e.g. lycopene, lutein, alpha-carotene, beta-carotene, beta-cryptoxanthin, zeaxanthin), calcium, iron, zinc, copper, iodine, selenium, thiamine, riboflavin, vitamin B6, vitamin B12, folic acid, vitamin C, vitamin D, vitamin A, arachidonic acid, or any mixtures thereof.

In a preferred embodiment of the invention, the nutrients are a mixture of docosahexaenoic acid (DHA) and alpha-linolenic acid, optionally with carotenoids.

The composition of the invention further comprises at least one probiotic. A probiotic may be defined as a live microbial feed supplement which beneficially affects the host animal by improving its intestinal microbial balance. The probiotic micro-organisms considered by this invention can include any probiotic selected form the group comprising of Bifidobacterium, Lactobacillus, Streptococcus, Enterococcus and Saccharomyces or mixtures thereof, preferably selected from the group consisting of Bifidobacterium longum, Bifidobacterium lactis, Lactobacillus acidophilus, Lactobacillus rhamnosus, Lactobacillus paracasei, Lactobacillus johnsonii, Lactobacillus plantarum, Lactobacillus salivarius, Lactobacillus reuteri, Enterococcus faecium, Streptococcus sp. and Saccharomyces boulardii or mixtures thereof. More preferably the probiotic is selected from the group comprising of Lactobacillus rhamnosus CGMCC 1.3724 (nick name NCC4007 and LPR), Bifidobacterium lactis CNCM I-3446 sold inter alia by the Christian Hansen company of Denmark under the trade mark Bb12 (nick mane NCC2818), Bifidobacterium longum ATCC BAA-999 sold by Morinaga Milk Industry Co. Ltd. of Japan under the trade mark BB536, Lactobacillus paracasei CNCM I-2116 (nick name NCC2461 and ST11), Lactobacillus johnsonii CNCM I-1225 (nick name NCC533 and La1), Lactobacillus fermentum VRI 003 sold by Probiomics (Australia), under the trademark PCC, Bifidobacterium longum CNCM I-2170, Bifidobacterium longum CNCM I-2618, Bifidobacterium breve sold by Danisco (Denmark) under the trade mark Bb-03, Bifidobacterium breve sold by Morinaga (Japan) under the trade mark M-16V and the strain of Bifidobacterium breve sold by Institut Rosell (Lallemand) (Canada) under the trade mark R0070, Lactobacillus paracasei CNCM I-1292, Lactobacillus rhamnosus ATCC 53103 obtainable inter alia from Valio Oy of Finland under the trade mark LGG, Enterococcus faecium SF 68, and mixtures thereof. A preferred probiotic is Lactobacillus rhamnosus CGMCC 1.3724. Another preferred probiotics is Lactobacillus reuteri, especially Lactobacillus reuteri ATCC 55730, ATCC PTA 6475, ATCC PTA 4659 and ATCC PTA 5289, and more particularly Lactobacillus reuteri ATCC 55730 and L. reuteri DSM 17938 obtainable from BioGaia AB (Kungsbroplan 3A Stockholm, Sweden).

Preferably, the composition provides a probiotic amount equivalent to between 10³ and 10¹⁰ cfu/g of composition (cfu=colony forming unit) or per day. This expression includes the possibilities that the bacteria are live, inactivated or dead or even present as fragments such as DNA or cell wall materials. In other words, the quantity of bacteria which the formula contains is expressed in terms of the colony forming ability of that quantity of bacteria as if all the bacteria were live irrespective of whether they are, in fact, live, inactivated or dead, fragmented or a mixture of any or all of these states. Preferably the composition provides a probiotic amount equivalent to between 10⁴ to 10⁹ cfu/g of composition or per day, even more preferably an amount equivalent to between 10⁶ and 10⁸ cfu/g of composition or per day.

Thus, a preferred composition according to the invention comprises Lactobacillus reuteri, docosahexaenoic acid (DHA) and alpha-linolenic acid.

The nutrient is preferably present in the compositions of the invention in an amount between 0.01% and 10% (w/w) of the composition and/or such as to provide between 10% and 100% of the daily recommended dosage. More specifically, the vitamins may be present in the composition such as to deliver between 5% and 100%, preferably between 20% and 80% of the daily recommended dosage for infants and children of the target age.

The composition may contain a source of lipids, preferably polyunsaturated fatty acids (PUFA). The lipid source may be any lipid or fat which is suitable for use in infant formulas. Preferred fat sources include low erucic rapeseed oil, soy oil, single cell oil, fish oil, MCT oil, palm olein, high oleic sunflower oil, sunflower oil and high oleic safflower oil. The essential fatty acids linoleic and α-linolenic acid may be present in the composition. In total, the fat content is preferably such as to contribute between 30 to 100% of the total energy of the supplement. The fat source preferably has a ratio of n-6 to n-3 fatty acids of about 1:20 to about 15:1; for example about 1:1 to about 10:1.

The polyunsaturated fatty acids (PUFA) may be present in an amount to provide a dose between 0-200 mg/day, more preferably 10 to 100 mg/day, even more preferably 20 to 65 mg/day.

The compositions of the invention may further comprise at least one prebiotic. A prebiotic may be defined as a non-digestible food ingredient that beneficially affects the host by selectively stimulating the growth and/or activity of one or a limited number of bacteria in the colon, and thus improves host health.

The prebiotic may be selected from N-acetylated oligosaccharide, neutral oligosaccharides or acidic oligosaccharides or any mixtures thereof.

N-acetylated oligosaccharides are characterized by the presence of an N-acetyl residue and include N-acetyl-lactosamine, N-acetyl-galactosaminyl glucose and N-acetyl-galactosyl lactose.

Neutral oligosaccharides are those oligosaccharides which have no charge. Apart from N-acetylated oligosaccharides, examples of neutral oligosaccharides include galacto-oligosaccharides, fructo-oligosaccharides, and fucosyl-oligosaccharides (e.g. fucosyllactoses, fucosylated lactosamine-lactoses, etc). Preferably, the neutral oligosaccharide include β-galacto-oligosaccharides (β-GOS).

Acidic oligosaccharides are those which have a charge. Preferred acidic oligosaccharides include sialylated oligosaccharides. These are characterized by one or more residues of N-acetylneuraminic acid, such as 3′- and 6′-sialyllactose (SL) and sialyl-lacto-N-tetraose. Other acidic oligosaccharides include those containing residues of uronic acid.

In an embodiment, the prebiotic is preferably selected from a human milk oligosaccharide. Preferably, the human milk oligosaccharide is selected from sialylated oligosaccharide, fucosylated oligosaccharide, or any mixtures thereof.

Preferably, an embodiment of the prebiotic comprises an oligosaccharide produced from glucose, galactose, xylose, maltose, sucrose, lactose, starch, xylan, hemicellulose, inulin, or a mixture thereof. More preferably the oligosaccharide comprises fructooligosaccharide. Most preferably the prebiotic comprises a mixture of fructooligosaccharide and inulin. Preferably this mixture comprises PREBIO1® or a mixture of commercially available RAFTILOSE® and RAFTILINE®.

Preferably, an embodiment of the prebiotic comprises about 50% to about 90% fructooligosaccharide. More preferably it comprises about 60% to about 80% fructooligosaccharide. Most preferably it comprises about 70% fructooligosaccharide.

Preferably, an embodiment of the prebiotic comprises about 10% to about 50% inulin. More preferably it comprises about 20% to about 40% inulin. Most preferably it comprises about 30% inulin.

When used, the prebiotic is preferably present in the compositions of the invention in an amount to provide a dose of 0.1-7 g/day more preferably 0.2 to 6 g/day, even more preferably 0.5 to 3 g/day.

The composition of the invention may be in the form of liquid drops, a gel, a cream, a powder etc.

In one embodiment the composition is in a liquid form at room temperature. It may comprise a liquid oil matrix. The liquid oil matrix serves both as a carrier for the composition and as a matrix able to induce a satisfactory preservation of the probiotic. The oil matrix may contain triglycerides, preferably medium chain triglycerides. The oil matrix is a food grade oil or food grade oil mix and may comprise low erucic rapeseed oil, soy oil, single cell oil, fish oil, palm oil, high oleic sunflower oil and/or sunflower oil. The oil matrix can comprise 50% or more, 70% or more, 90% or more 95% or more of the total composition.

When the composition is in a dry powder form, the composition can be provided at between 50 mg and 12 g per day, preferably between 0.2 g and 5 g per day.

When the composition is in a liquid form, the composition can be provided between 0.05 ml and 6 ml per day, preferably between 0.2 ml and 3 ml per day.

The compositions of the invention may be prepared in any suitable manner. For example, the nutrients can be mixed together in a dry form and suspended in an oil matrix. Freeze-dried or spray-dried probiotics can be added.

If it is desired to produce a liquid composition, the mixture is filled into suitable containers, preferably aseptically. In one embodiment, the composition may also be retorted in a container, preferably before the addition of the probiotic. Suitable apparatus for carrying out filling of this nature is commercially available. The liquid composition may be in the form of a ready-to-feed composition or in the form of a concentrate. The concentrate may also be administered directly to the infant, depending on the dosage required.

If it is desired to produce a powdered composition, the liquid mixture is preferably transferred to a suitable drying apparatus such as a spray drier or freeze drier and converted to powder. The powder should have a moisture content of less than about 5% by weight.

If the composition is a powder, it is preferably reconstituted in a liquid prior to administration to the infant.

The composition of the invention offers the advantage that it ensures a supply of nutrients which may vary in human breast milk.

In addition, due to the presence of probiotics, a number of benefits are also observed. The benefits associated with the presence of probiotics in the present compositions encompass controlling or preventing colic, reducing regurgitations, improving gut motility, abdominal distension, gut pain, and intestinal transit, reducing constipation, diarrhea, infections and allergies, improving immunity and sleep, preventing inflammation later in life.

It has also been found that due to the presence of nutrients in the present compositions, the effects of the probiotics are enhanced. Thus, a synergy between the presence of probiotics and the nutrients is achieved by the present compositions. For example the stability of the probiotic in the composition may be enhanced by the presence of said nutrient. Alternatively or additionally the bioavailability of the probiotic and/or of the nutrient can be enhanced by the synergy.

The compositions may be delivered to the infant in single dosage units. This has the advantage of preserving stability and preventing contamination of the compositions.

In an embodiment, the invention pertains to a method for maintaining exclusive breast-feeding in infants by feeding said infants a composition according to the invention.

Thus, the composition of the invention may be administered to the infant at least once daily. It may also be administered at each feeding session and/or in between feeding sessions.

An embodiment of the invention relates to the use of a composition described herein for preserving exclusive breast feeding in infants. The advantages of using the present composition are therefore that the infant does not need any other source of food such as infant formula to meet its dietary requirements. This also presents a financial advantage to the mother.

The compositions of the invention may be used in a method for improving the health of breast-fed infants or pets, such as e.g. cats or dogs. Thus a composition according to the invention for use in improving the health of breast-fed infants forms part of the invention.

The improvement of health is achieved by decreasing or preventing nutritional deficits and colics, reducing regurgitation, reducing allergies, reducing atopic diseases, fighting infections, improving cognitive development, improving gut maturation, improving gut motility, reducing abdominal distension and gut pain, improving intestinal transit, preventing or reducing constipation and diarrhea, improving neonatal immunity, boost immunity later in life, controlling adipogenesis, preventing overweight later in life, preventing inflammation later in life, improving sleep pattern, improving visual acuity, etc.

In order to improve the health of breast-fed infants, the composition is preferably administered to said infant daily.

In another aspect of the invention, a method of preparing a nutritional supplement composition for breast-fed infants is provided.

The method comprises the first step of selecting at least one nutrient from the most variable nutrients in human breast milk.

This can be easily achieved by a person of skill in the art simply by comparing the constitution of human breast milk across a population of women and selecting those nutrients which vary the most.

The at least one nutrient is preferably selected from vitamins, provitamins, carotenoids, polyunsaturated fatty acids, minerals, amino acids. In a particular embodiment, these nutrients can also include peptides.

Thus, the nutrient may be selected from any of docosahexaenoic acid (DHA), alpha-linolenic acid, carotenoids (e.g. lycopene, lutein, alpha-carotene, beta-carotene, beta-cryptoxanthin, zeaxanthin), calcium, iron, zinc, copper, iodine, selenium, thiamine, riboflavin, vitamin B6, vitamin B12, folic acid, vitamin C, vitamin D, vitamin A, arachidonic acid, or any mixtures thereof.

Said nutrient is then incorporated in a nutritional matrix to form a nutritional supplement composition. This is carried out by typical methods used in the art.

By “nutritional matrix” is meant any ingestible matrix. The matrix may comprise any of oils, milk powder or other matrix-building ingredients.

The resulting nutritional supplement composition is thus enriched in at least one particular nutrient and offers the advantage that it can be used in exclusively breast-fed infants in order to compensate for possible nutrient sub-deficiencies.

In a preferred embodiment, the nutritional supplement composition obtained by the method of the invention further comprises a probiotic. The probiotic may be any of those described in the present application.

The present invention is further illustrated by means of the following non-limiting examples.

### EXAMPLES

A composition is made having the following specific nutrients in a oil matrix. The oil matrix is a conventional edible oil for infants. A daily dose of 1 ml is recommended.
amounts of individual ingredients in the composition:
   1) L. reuteri DSM 17938: 10⁸ cfu per daily dose
   2) DHA: 20 mg Docosahexaenoic Acid (i.e. 50 mg DHASCO oil) per daily dose.
   3) ALA: 45 mg Alpha-Linolenic Acid (i.e. 500 mg canola oil (low erucic rapeseed oil) per daily dose
   4) Carotenoids: per daily dose:
      Lutein: 2.8 micro-g
      Beta-carotene: 1.6 micro-g
      Lycopene: 1.6 micro-g

The effect of probiotic L. reuteri is illustrated in the context of infant formula in the following publications: 1) Savino et al demonstrating anticolicky effects “Savino F, Pelle E, Palumeri E, Oggero R, Miniero R. 2006. Lactobacillus reuteri (American Type Culture Collection Strain 55730) versus simethicone in the treatment of infantile colic: a prospective randomized study. Pediatrics. 2007 January;119(1):e124-30”; 2) Indrio et al. demonstrating an improved gastrointestinal motility and bowel habits, “Indrio F, Riezzo G, Raimondi F, Bisceglia M, Cavallo L, Francavilla R. 2008. The effects of probiotics on feeding tolerance, bowel habits, and gastrointestinal motility in preterm newborns. J Pediatrics 152: 801-6”

1. Nutritional supplement composition for breast-fed infants or pets comprising at least one probiotic and at least one nutrient selected from the nutrients in human breast milk.

2. Composition according to claim 1, wherein the nutrient is selected from the group consisting of vitamins, provitamins, carotenoids, polyunsaturated fatty acids, long chain polyunsaturated fatty acids, minerals, amino acids, peptides, and mixtures thereof.

3. Composition according to claim 1, wherein the nutrient is selected from the group consisting of docosahexaenoic acid (DHA), arachidonic acid (ARA), alpha-linolenic acid, carotenoids such as e.g. lycopene, lutein, alpha-carotene, beta-carotene, beta-cryptoxanthin, zeaxanthin, calcium, iron, zinc, copper, iodine, selenium, thiamine, riboflavin, vitamin B6, vitamin B12, folic acid, vitamin C, vitamin D, vitamin A, and mixtures thereof.

4. Composition according to claim 1, wherein the nutrient comprises a mixture of docosahexaenoic acid and alpha-linolenic acid.

5. Composition according to claim 1, wherein the probiotic is selected from the group consisting of a lactobacillus, a bifidobacterium, an Enterococcus, a saccharomyces, a streptococcus, and mixtures thereof.

6. Composition according to claim 1, comprising at least one prebiotic.

7. Composition according to claim 1, wherein the composition is a form selected from the group consisting of liquid drops, gel, cream, and powder.

8. Composition according to claim 1, wherein the probiotic is present in an amount to provide a daily dose of between 10³ to 10¹⁰ cfu/day.

9. Composition according to claim 1, wherein at least one nutrient is present in an amount such to provide between 5 and 100% of the daily recommended dosage for infants and children of the target age.

10. Composition according to claim 1, having a caloric density of 6.5 kcal/g.

11. Composition according to claim 1, which provides a protein dose of less than 1.5 g/100 kcal.

12. The composition according to claim 1, having a fat value of more than 5 g fat/100 kcal.

13. The composition according to claim 1, wherein the fat content comprises more than 50% of the caloric value of the composition.

14. The composition according to claim 1, having a fat value of less than 1 g fat/100 kcal.

15. A method for preserving exclusive breast feeding of infants comprising the steps of administering a composition comprising at least one probiotic and at least one nutrient selected from the nutrients in human breast milk to an infant.

16. A method for improving the health of breast-fed infants comprising the steps of administering a composition comprising at least one probiotic and at least one nutrient selected from the nutrients in human breast milk to an infant.

17. Method according to claim 16, wherein the improvement of health is achieved by achieving a result selected from the group consisting of reducing or preventing nutritional deficits, decreasing or preventing colics, reducing regurgitation, reducing allergies, reducing atopic diseases, fighting infections, improving cognitive development, improving gut maturation, improving gut motility, reducing abdominal distension and gut pain, improving intestinal transit, preventing or reducing constipation and diarrhea, improving neonatal immunity, boost immunity later in life, controlling adipogenesis, preventing overweight later in life, preventing inflammation later in life, improving sleep pattern, and improving visual acuity.

18. Composition according to claim 16, wherein the composition is administered to the breast-fed infant daily.

19. Method of preparing a nutritional supplement composition for breast-fed infants or pets comprising the steps of:
selecting at least one nutrient from the most variable nutrients in human breast milk and
incorporating the nutrient in a nutritional matrix to form a nutritional supplement composition.

20. Method of claim 19, wherein the nutritional supplement composition comprises a probiotic.

21. Composition according to claim 1, wherein the probiotic is selected from the group consisting of Lactobacillus rhamnosus, Lactobacillus paracasei, Lactobacillus reuteri, Lactobacillus acidophilus, Lactobacillus johnsonii, Lactobacillus plantarum, Lactobacillus salivarius, Streptococcus thermophilus, Bifidobacterium longum, Bifidobacterium lactic, Enterococcus faecium, Streptococcus sp. and Saccharomyces boulardii and mixtures thereof.
